(19) United States
(12) Patent Application Publication
(10) Pub. No.: US 2002/0192206 A1
(43) Pub. Date: Dec. 19, 2002

(54) Methods and compositions for angioproliferative disorder treatment

(76) Inventors: Emil V. Kozarov, Gainesville, FL (US); Ann Progulske-Fox, Gainesville, FL (US)

Correspondence Address: MCDONNELL BOEHNEN HULBERT & BERGHOFF, 300 SOUTH WACKER DRIVE, SUITE 3200, CHICAGO, IL, 60606, US

(21) Appl. No.: 09/849,115

(22) Filed: May 5, 2001

(51) Int. Cl. A61K 48/00; A61K 38/48

(52) U.S. Cl. 424/94.63; 514/44

(57) ABSTRACT

An invention is provided whereby methods and compositions having angiostatic activity are utilized to treat angioproliferative disorders, to prevent conception, and to treat a wide variety of pathologies in which it is desirable to limit the production of new vasculature. Specifically, compositions containing proteinases derived from the pathogen Porphyromonas gingivalis capable of treating cancer through disruption of cell-cell and cell-matrix adhesion bonds associated with malignant tumor proliferation are disclosed.

### FIELD OF THE INVENTION

This invention is generally directed to compositions and methods of their use for treatment of angioproliferative disorders. Specifically, compositions containing proteases, peptides related to the HagA gene product and fragments thereof derived from the pathogen Porphyromonas gingivalis are disclosed which are capable of treating cancer through disruption of cell-cell and cell-matrix adhesion bonds associated with malignant tumor proliferation.

### BACKGROUND INFORMATION

Cancer is the second leading cause of death in the United States, accounting for over one half million deaths per year. (National Vital Statistics Report, 1998 Vol. 48, No. 11). The total economic cost associated with cancer has been estimated to be over $100 billion dollars annually. (Brown, M. L. et al In Cancer Epidemiology and Prevention 1996). There is currently no cure for the disease, but several lines of research appear promising. Of these, research directed at preventing angiogenesis offers the most hope.

Angiogenesis involves a complex biochemical cascade of events that leads to new blood vessel formation in developing tumors. For cells to survive, each must have some communication, direct, or indirect with the existing vasculature in order to obtain nutrients and oxygen, and to offload metabolic waste products. Active vascularization is normally observed following injury to a tissue, during development, or in response to ovulation in females. However, abnormal rapid proliferation of blood vessels is also observed in areas where cancerous masses have developed. Because the rate of cell mass growth is limited by the degree of vascularization present, cancer cells release chemical substances into the surrounding environment to induce nearby, nourishing blood vessels to grow toward the proliferating cancerous cell mass. For example, angiopoietin-1, basic fibroblast growth factor (bFGF), vascular endothelial growth factor (VEGF) and other substances are released from cancer cells to coax surrounding blood vessels to grow collateral vessels toward the tumor. Once vascularized, a tumor mass may grow locally, or may metastasize and begin to spread through the bloodstream and lymphatic system to other parts of the body, causing significant damage. For example, vascularization of the retina in diabetic retinopathy can lead to retinal detachment resulting in blindnessMetastasis is a hallmark of malignancy, which in extreme cases may lead to rapid death of an individual. Ironically, some tumors may also secrete angiostatic substances to inhibit tumor growth. (Chen et al, Cancer Res. 1995 55, 4230-4233; O'Reilly, Cell , Jan. 24, 1997 (88):277-285). Thus, it appears that in healthy individuals; angiogenesis associated with tumor growth may be regulated by a fine balance between the release of angiogenic factors and the release of angiostatic factors. It is believed that by blocking the process of angiogenesis, tumor growth can be suspended, which in turn would lead to cancer remission.

Most early research directed at preventing angiogenesis involved exposing various cell lines to angiostatic compounds and assessing the degree of proliferation either in vivo or in vitro. The National Cancer Institute, for example, uses proliferation, migration and cord formation assays in HUVEC cells for its anti-angiogenisis testing. Several angiostatic agents that function to prevent the proliferation of cancer cells have been isolated and tested. For example, administration of Angiostatin has been shown to suppress vascular endothelial cell proliferation, thereby reducing the size and lethality of tumors (Folkman J., Forum Genova Jul.-Dec. 9, 1997 (3 Suppl.3):59-62). Recombinant Endostatin (baculovirus) has been used to inhibit the proliferation of bovine capillary endothelial cells. (O'Reily et al. Cell Oct. 21, 1994 ;79(2):185-8). Until recently, angiostatic compounds have included only those substances capable of preventing proliferation of cells. However, a growing body of evidence supports the view that agents that inhibit proliferation via cellular detachment from tumor masses perform an analogous function.

Recent studies have been directed at interfering or disrupting the mechanisms involved in cell-cell or cell-matrix binding as a means to reduce or eliminate cancerous growth. Researchers have known for some time that cells will not proliferate if they are not first attached to a surface. For example, impaired cell-matrix contact leads to anoikis (epithelial apoptosis) (Vitale, M. et al. FEBS Lett 1999 462(1-2:57-60), (Attwell, S. et al. Oncogene 2000 19(33):3811-5), (Rosen, K. J Cell Biol 2000 149(2):447-56), (Rytomaa, M, et al. Curr Biol 1999 9(18):1043-6) or endothelial apoptosis (Erdreich-Epstein, Cancer Res Feb. 1, 2000; 60(3):712-21). This anchorage dependence is mediated, in part, by cell surface molecules known as integrins. The role of integrins in tumor growth and metastisis has been recognized in the literatureErdreich-Epstein et al. Cancer Res. Feb. 1, 2000; 60(3):712-21demonstrated that inhibition of integrin dependent endothelial cell anchorage to vitronectin, induced apoptosis in bovine brain endothelial cells, which in-turn resulted in disruption of tumor angiogenesis and inhibition of tumor growth. Lee, J W and R. L. Juliano. Mol Biol Cell Jun. 11, 2000 (6): 1973-87demonstrated that integrin protects intestinal epithelial cells from apoptosis. Further, attachment of squamous cancer cells to Matrigel has been reduced by the integrin substrate RGD (Kawahara, E. J Cancer Res Clin Oncol 1995 121 (3):133-40) Integrin α5β3 is known to protect intestinal epithelial cells from apoptosis (Lee, W Mol Biol Cell 2000 11(6): 1973-87). Integrin α5β3 (vitronectin receptor) can potentiate the effects of insulin and certain other growth factors and the α5β1 integrin (fibronectin receptor) supports cell survival in serum-free cultures by up-regulating the anti-apoptosis protein Bcl-2 (Ruoslahti, E. Kidney Int 1997 51(5):1413-7). (Brassard, D et al. 1999 Exp Cell Res 251 (1):33-45). Detachment-induced apoptosis is responsible for the antiproliferative effects of EGF in breast cancer cells (Kottke, T. J. et al. J. Biol Chem 1999 274(22):15927-36).

Antiangiogenic approach is the most recent and promising avenue in cancer treatment. Agents capable of blocking vascularization of neoplastic tissue may prevent subsequent growth of the transformed tissue and may lead to existing tissue remission. Antiangiogenic activity has been detected for several endogenous factors For example, an antiproliferative/cytotoxic effect was demonstrated for combrestatin A-4 disodium phosphate (CA4DP) against proliferating endothelial cells, but not cells that were quiescent prior to and during drug exposure (Dark, G. G. Cancer Res. May 15, 1997 ; 57 (10):1829-34) Also, WIPO PCT publication no.WO9960984 issued to Fortier disclosed a purified human PSA compound capable of inhibiting FGF-2 and VEGF-stimulated migration, thereby preventing endothelial cell proliferation in bovine endothelial cell and human endothelial cell lines (HMVEC-d and HUVEC). The degree to which endothelial cell invasion was inhibited confirmed that PSA has anti-angiogenic properties. Taddei, L. et al Biochem Biophys Res Commun. Sep. 24, 1999: 263 (2): 340-5. measured the cell detachment capacity of Endostatin by allowing CVEC cells to attach to a substrate and subsequently exposing them to a composition containing Endostatin. The proliferation of CVEV cells was quantified by total cell number that remained attached to the substrate. The researchers concluded that cell detachment is linked to proliferation. Further, somatostatin has been shown to control Kaposi's sarcoma tumor growth through inhibition of angiogenesis (Albini, A. Faseb J 1999 13(6):647-55). Kotke, T. J. et al. J. Biol Chem. May 28, 1999;274(22): 15927-36 examined Epidermal Growth Factors (EGF) which act in cell proliferation and apoptosis and found that detachment-induced apoptosis in breast cancer cells exposed to chemotherapeutic agents is responsible for the known anti-proliferative effects of EGF. Further, angiostatin is known to inhibit endothelial and melanoma cellular invasion by blocking matrix-enhanced plasminogen activation (Stack, M. Biochem J 1999 340 (pt1):77-84). Accordingly, the preceding studies provide directevidence that detachment is linked to proliferation, and that materials capable of causing cell detachment through interruption of cell-cell or cell-matrix bonds function as anti-proliferative agents.

For many years, scientists have been in search of therapeutics which may be used to prevent periodontal diseases, including gum infections and tooth decay. One organism that has been identified as a potential etiologic agent of such pathologies as gingivitis and periodontal disease is the pathogen Porphyromonas gingivalis. Sequences from that pathogen have been cloned and sequenced and the role of that pathogen has been elucidated. Examples of such work may be found in U.S. Pat. Nos. 5,824,791 and 5,830,710, both of which are hereby incorporated herein by reference in their entirety. Surprisingly, the instant inventors have found that agents derived from the pathogen P. gingivalis may also be used as effective anti-angioproliferative agents, through their ability to disrupt cell-cell or cell-matrix bonds, and thereby prevent vascularization of tissue. As there is no known cure for cancer currently available, a need exists for angiostatic agents capable of treating cancer development and preventing its progression. The systemic or local application of the protease derived from P. gingivalis provided according to this invention constitutes an entirely new method and composition for treatment of both growing and existing tumors.

### SUMMARY OF THE INVENTION

The present invention discloses compositions of matter, and methods of their use, capable of disrupting endothelial tissue growth and proliferation. Compositions according to this invention contain proteases and peptides related to the Hemagglutinin A (HagA) gene product isolated from the pathogen Porphyromonas gingivalis. The method involves local or systemic application of P. gingivalis polypeptide to reach a targeted tissue. This novel method of treating cancer utilizes P. gingivalis derived proteolytic compounds to inhibit angiogenesis associated with malignant tumor proliferation by disrupting endothelial layer cell-cell and cell-matrix adhesion bonds. Use of these compounds and method has advantages over conventional treatments such as chemotherapy, because it targets only growing vessels, while leaving intact vessels unaffected.

Accordingly, it is one object of this invention to provide novel compositions of matter for treatment of tumors.

A further object of this invention is to provide methods for treatment of tumors.

A further object of this invention is to provide methods of making Porphyromonas gingivalis derived compounds for treatment of tumors.

A further object of this invention is to provide angiostatic compositions.

A further object of this invention is to provide methods of using angiostatic agents derived or related to Porphyromonas gingivalis nucleic acids, peptides and polypeptides.

Yet a further object of this invention is to provide compositions capable of disrupting cell-cell and cell-matrix adhesion bonds.

Yet a further object of this invention is to provide methods for disrupting cell-cell and cell-matrix adhesion bonds.

Yet a further object of this invention is to provide compositions capable of preventing implantation of a fertilized ovum.

Still a further object of this invention is to provide methods of contraception.

Yet a further object of this invention is to provide a composition for treating or preventing ocular retinopathy, retrolental fribroplasia, psoriasis, angiofibromas, endometriosis, hemangioma, rheumatoid arthritis, and capillary proliferation within atherosclerotic plaque.

Yet a further object of this invention is to provide a method of treating or preventing ocular retinopathy, retrolental fribroplasia, psoriasis, angiofibromas, endometriosis, hemangioma, rheumatoid arthritis, and capillary proliferation within atherosclerotic plaque.

Further objects and advantages of this invention will be appreciated from a review of the complete disclosure and the appended claims.

### BRIEF DESCRIPTION OF THE FIGURES

FIG. 1 is a bar graph showing percent detachment of active Human Umbilical Vein Endothelial Cells (HUVEC) after 24 hours of treatment with a protein extract from P. gingivalis. Represented are mean values from triplicate experiments.

FIG. 2 is a bar graph showing percent detachment of active Human Umbilical Vein Endothelial Cells (HUVEC) after 48 hours of treatment with a protein extract from P. gingivalis.

FIG. 3 is a bar graph showing percent detachment of quiescent Human Umbilical Vein Endothelial Cells (HUVEC) after 24 hours of treatment with a protein extract from P. gingivalis.

FIG. 4 is a bar graph showing percent detachment of quiescent Human Umbilical Vein Endothelial Cells (HUVEC) after 48 hours of treatment with a protein extract from P. gingivalis.

FIG. 5 is a bar graph showing percent detachment of active human non-small cell lung carcinoma cell line (A549) after 24 hours of treatment with a protein extract from P. gingivalis.

FIG. 6 is a bar graph showing percent detachment of active human non-small cell lung carcinoma cell line (A549) after 48 hours of treatment with a protein extract from P. gingivalis.

FIG. 7 is a bar graph showing percent detachment of quiescent human non-small cell lung carcinoma cell line (A549) after 24 hours of treatment with a protein extract from P. gingivalis.

FIG. 8 is a bar graph showing percent detachment of quiescent human non-small cell lung carcinoma cell line (A549) after 48 hours of treatment with a protein extract from P. gingivalis.

FIG. 9 is a bar graph showing the reduction in human vascular endothelial cell migration after exposure to a protein extract from P. gingivalis over a 24-hour period.

FIG. 10 is a bar graph showing detachment of human non-small cell lung carcinoma cell line (A549) after treatment with a proteinase extract of P. gingivalis alone and with a host Bacteroides fragilis expressing PrTP protease from P. gingivalis.

FIGS. 11a-d generally shows active fragments of the HagA gene product. FIG. 11a shows affinity-purified recombinant HagArunning at 100 kDa. FIG. 11b shows a Western blot of the same protein probed with Mab specific for an epitope in HagA FIG. 11c shows a Western immunoblot assay for immunodection of HagA—binding proteins from epithelial cell lines. FIG. 11d shows the same test, but for endothelial (HCAEC) human cell lines. immunodetection assay for detection of HagA binding proteins.

FIG. 12 is a table showing the degree of proliferation inhibition of a HUH7 cell line exposed to P. gingivalis and E. coli extract, and to P. gingivalis cells in the presence or absence of inhibitors.

FIGS. 13a-e generally show the results of reactivity of a HUH7 cell line with anti-occludin antibodies. FIG. 13a depicts non-treated control HUH7 cells. FIG. 13b depicts the results of treatment with P. gingivalis extract. FIG. 13c depicts the results of treatment with P. gingivalis extract in the presence of the inhibitor TLCK. FIG.13d depicts results of treatment with heat-treated P. gingivalis extract. FIG. 13e depicts the results of a control treatment with E. coli extract.

FIGS. 14a-d generally shows the results of reactivity of a HUH7 cell line with anti-pan cadherin antibody. FIG. 14a depicts non-treated, control HUH7 cells. FIG. 14b depecits the results of treatment with P. gingivalis extract. FIG. 14c depicts the results of treatment with P. gingivalis extract in the presence of inhibitor TLCK. FIG. 14d depicts the results of treatment with heat inactivated P. gingivalis extract.

FIGS. 15a-c generally shows the proliferation inhibition of a HUVEC polarized cell line.

FIG. 15a depicts non-treated, control polarized human endothelial cells, ECV-304.

FIG. 15b depicts the results of ECV-304 cells treated luminally with 60% fraction of P. gingivalis culture liquid proteins. FIG. 15c depcists the results of ECV-304 cells treated basolaterally with 60% fraction of P. gingivalis culture liquid proteins.

### DETAILED DESCRIPTION OF THE INVENTION

Current antiangiogenic strategies include blocking the ability of the endothelial cells to break down the surrounding matrix, inhibiting normal endothelial cells directly, blocking factors that stimulate angiogenesis, or blocking the action of integrins. The present invention provides a novel strategy of cancer treatment using protein, peptide and nucleic acid sequences of Porphyromonas gingivalis to treat or prevent angioproliferative disorders.

In U.S. Pat. No. 5,830,710, (the '710 patent) protein, peptide and nucleic acid sequences of Porphyromonas gingivalis genes were disclosed. Methods of use of such materials for the detection of the pathogen were disclosed and claimed. Additionally, methods of diagnosing infections caused by the pathogen, and methods of use of such materials to treat and prevent such infections, including through use as vaccines, were also disclosed. The disclosure of U.S. Pat. No. 5,830,710 is hereby incorporated herein by reference, as if fully set forth herein.

In U.S. Pat. No. 5,824,791, (the '791 patent) additional sequences and methods of use thereof, including as vaccines, cells transformed with P. gingivalis sequences and methods of production of such compositions were further disclosed. The disclosure of U.S. Pat. No. 5,824,791 is hereby incorporated herein by reference, as if fully set forth herein.

As used herein, the term “proteinase” is intended to mean a protein, peptide, active site, analog or homologue thereof, which exhibits angiostatic activity.

The term “pharmaceutically effective amount” as used herein is intended to mean an amount of a compound such that through routine experimentation, based on disclosure and guidance provided herein, a dosage of the relevant compound or activity may be determined such that the specified functional result is achieved.

The term “angiostatic” as used herein is intended to mean any substance capable of preventing endothelial cell proliferation, promoting cellular detachment, and inhibiting migration of endothelial cells, thereby blocking the formation of new blood vessels, or destroying existing blood vessels feeding tumors.

The term “angiostatically effective amount” as used herein is intended to mean an amount of a compound such that through routine experimentation, based on disclosure and guidance provided herein, a dosage of the relevant compound or activity may be determined such that an anti-angiogenic effect, including but not limited to prevention of new vessel formation, inhibition of vascular proliferation, disruption of vascular endothelium, inhibition of endothelial cell migration, inhibition of tumor enlargement, decrease in tumor mass, and related biological processes associated with angiogenesis and vascular supply to a particular biological organ or location is achieved.

The term “protease” as used herein is intended to include any proteolytic activity that produces an anti-angiogenic or anti-angioproliferative effect. The term is intended to imply the ability of an enzyme, natural or recombinant, or a compound that exhibits an enzyme-like activity, to disrupt cell-cell adhesion and cell-matrix adhesion bonds, particularly in the vasculature, but also in the tumor mass.

The term “bacterial” as used herein is intended to mean Porphyromonas gingivalis (P. gingivalis) and related bacterial organisms, as well as other bacteria which express proteases having activities similar to that expressed by P. gingivalis wherein the activity is effective as an angiogenesis inhibitor.

The terms “angiogenesis” and “angioproliferative” as used herein are intended to imply conditions wherein rapid, usually uncontrolled and often pathologic development of vascular supply to a particular organ or biological site occurs, as in the development of a tumor. A generally non-pathologic condition where such angioproliferation occurs, and to which this invention is likewise directed as needed, occurs upon implantation of a fertilized embryo.

The present inventors have discovered that, surprisingly, sequences derived from or related to sequences disclosed in the '710 and '791 patents may be used to treat or prevent angioproliferative disorders, including but not limited to melanoma, sarcoma, and carcinomas of the breast, colon, lung and prostate. Additional pathologies susceptible to treatment according to the present invention include ocular retinopathy, retrolental fribroplasia, psoriasis, angiofibromas, endometriosis, hemangioma, rheumatoid arthritis, and capillary proliferation within atherosclerotic plaque. Furthermore, due to the angiostatic activity of the compositions disclosed herein, use as a contraceptive to prevent implantation of fertilized ova is also contemplated herein.

Use of P. gingivalis peptides as angiostatic agent for tumor reduction and cancer treatment has not been discussed in the literature. Layman et al., (citation) discusses how P. gingivalis naturally produces a newly identified vascular disruption protein but does not disclose its use as an anti-tumor agent. Parramaesvarans, D. et al. 76^{th} General Session of IADR, 1998 Nice, France identified a 15 kDA adhesin domain from P. gingivalis that may be used in vascular disruption. However, the abstract is directed to the pathologic activity induced by this anti-angiogenic agent in periodontal disease, without focusing on the potentially therapeutic application of this protein as an anti-tumor or anti-angiogenic agent. Other studies have shown that bacterial extracts may be useful as angiostatic agents. For example, Phillips, J. R. (J. Periodontal Res. 1990 Nov; 25(6):339-46) demonstrated that a soluble sonic extract from Bacteroides gingivalis causes a dose dependent inhibition of gingival fibroblast growth, reduced cell attachment and altered cell morphology. Also, two angiogenesis inhibitors, tecogalan sodium and DS-4152, have been isolated from a sulfated polysaccharide produced by the bacterium Arthrobacter (Eckhardt, S. et al Ann Oncol 1996 7(5):491-6), (Baba, M. Aids 1994 8(1:43-8). However, no study has demonstrated that an extract from P. gingivalis functions in tumor prevention or destruction. ther patents have issued directed to modulating cell adhesion between tumor cells. For example, U.S. Pat. No. 6,110,747 discloses methods of using agents that inhibit occludin-mediated cell adhesion. U.S. Pat. No. 6,031,072 discloses compounds for modulating cadherin-mediated cell adhesion. U.S. Pat. No. 6,169,071 discloses methods for inhibiting cadhedrin-mediated cell adhesion. However, none of these patents disclose a method of treating cancer through protease-mediated CAM degradation.

Without wishing to be restricted to a specific mechanism of action, it is contemplated that the P. gingivalis organism inhibits angiogenesis, at least in part, through proteases produced by the bacterium that exhibit an angiostatic activity. These proteases disrupt cell-cell and cell-adhesion bonds critical to blood vessel formation and tissue proliferation. Interactions with tight junctions are not typical for bacteria. E-cadherin proteolysis has only been reported for BFT acting on BL side of intestinal epithelia (Wu, H et al. Proc Natl Acad Sci USA 1998 95(25): 14979-84), whereas the other well-studied bacterial toxins (tetanus, botulinum) act intracellularly. Clostridium difficile toxin A also permeabilizes ZO (Hecht, G. et al J Clin Invest 1988 82 (5):1516-24). Also, D. pteronyssinus protease acting from the lumenal side, has been shown to open the paracellular barrier in airway epithelia and lead to asthma (Wan, H. J Clin Invest 1999 104(1):123-33 1999). Further, inhibition of tumor cells attachment and chemotactic invasion has been correlated with the loss of E-cadherin (Irie, T. et al. Anticancer Res 1999 19(4b):3061-6). It is believed that the proteolytic enzymes of Porphyromonas gingivalis are the main tool for providing nutrients to these asaccharolytic bacteria and are important virulence factors. These enzymes have been shown to degrade basement membrane matrix proteins (Uitto, V. Oral Microbiology and Immunology 1988 3:97-102), (Smalley, J Arch Oral Biol 1988 33 (5):323-9). Further, purified P. gingivalis cysteine protease has been shown to disrupt the basement membrane of human carcinoma monolayer (Shah, H. J Periodontol 1992 63(1):44-51).

The protease isolated from P. gingivalis according to the present invention acts on cell surface adhesion molecules (CAM's), from the basolateral side of the endothelium. In one embodiment, these proteases inhibit angiogenesis by targeting integrin in cell-cell and cell-matrix adhesion bonds. Therapies directed at influencing integrin cell expression and function are presently being explored for inhibition of tumor growth, metastasis, and angiogenesis (Mizejewski, G. J. et al Proc Soc Exp Biol Med 1999 222(2): 124-38), (Velasco-Velazquez, M. A. Rev Invest Clin. 1999 51 (3):183-93), Recently, targeted disruption of fibronectin-integrin interactions in human gingival fibroblasts has been demonstrated by the RI protease of P. gingivalis W50 (Scragg, M. A. et al. Infect Immun 1999 67(4):1837-43). Other studies have examined the function of P. gingivalis extract on cell-cell and cell-matrix bonds. Cell detachment from each other and from the underlying surface correlates with the cysteine-dependent proteolytic activity of P. gingivalis (Johansson, A. Eur J Oral Sci 1998 106(4):863-71). β1-integrin, occludin and E-cadherin are targeted by P. gingivalis proteolytic activity in canine epithelial cells (Katz, J et al. Infect Immun 2000 68(3): 1441-9).

Here, the present inventors propose for a first time a method for tumor ablation therapy utilizing P. gingivalis endothelial layer disrupting activity. In addition, significant proliferation inhibition was also observed with lung and hepatoma tumor cell lines. In periodontal disease, this activity has been demonstrated to result in necrosis of the surrounding tissue. According to the present invention, the angiostatic activity of P. gingivalis proteins and peptides is localized, and extended to a model predictive of the ability of the compositions of matter disclosed herein to disrupt endothelial layers and basal membranes, and to prevent proliferation of new or aberrant vasculature. In order for tumors to grow, the existing vasculature must be coaxed to sprout collateral vessels to supply the neoplastic tissue. Blocking this process prevents the transformed tissue from proliferating, forcing the existing tumors into a state of remission. In addition, tumor ablation by disintegrating cell to cell bonds in the tumor itself is envisaged.

According to one embodiment of the present invention, a unique P. gingivalis protease which may be derived from the HagA gene is utilized as an angiostatic agent. Of particular importance to methods of use of this protease according to the present invention is the fact that vasculature supplying tumor tissues is aberrantly leaky. Regular blood vessels are well formed and have a well-developed adhesion system to keep them together, while blood vessels supplying tumors are poorly formed and extremely leaky (Herlyn, M. J. Immunother. May 22, 1999 (3): 185); see also: Hashizume H. et al Am J Pathol. 2000 April;156(4):1363-80). As a result, locally or systemically administered P. gingivalis proteinase, analogs thereof, the active site, subunit, peptidomimetics thereof, and similar proteases from other sources, natural or recombinant, leak out of the vasculature which supplies the tumor tissue. Access to the basolateral surface of the vasculature is thereby achieved, which results in disruption of the vasculature at that location, and hence dissipation of the thus isolated tumor. Thus, compounds disclosed herein can disrupt angiogenesis without affecting the integrity of normal blood vessels. Intravenous, topical, controlled release and other modes of administration of the protease according to this invention are contemplated. Similar methods of treating other angioproliferative conditions mentioned herein as well as other angioproliferative conditions suggested by those mentioned herein come within the scope of the present disclosure.

In another embodiment according to this invention, those skilled in the art will appreciate, based on the present disclosure, that methods for promoting angiostatis have implications for contraception. During pregnancy, the endometrial layer of the uterus become thickened and engorged with blood vessels upon implantation of a fertilized ovum. Without a well-developed vasculature, the fertilized ovum will not be sustained, and the endometrial layer will be sloughed-off in the form of menses, i.e., menstruation. In one embodiment of the present invention, therapeutic compositions for use as contraceptives are provided. In order to induce contraception, the internal vasculature of the uterus is contacted with a contraceptively effective amount of HagA, an analog or derivative thereof. The mode of achieving bioavailability of this compound for this and other angioproliferative conditions is either through systemic or localized administration.

In the case of a contraceptive utility, various modes of delivery may be contemplated, including, but not limited, to systemic delivery of doses of the P. gingivalis proteinase functionality at dosages non-toxic to the remainder of the organism. Intrauterine infusion for topical administration is also contemplated herein. In this manner contraceptive efficacy may be achieved through administration of the disclosed angiostatic agent.

Because of the specificity of this protease for rapidly developing vasculature, it is possible to define doses of the P. gingivalis proteolytic functionality which are non-toxic to the remainder of the organism, but which nonetheless provide a localized effect to achieve angiostasis where beneficial effects thereof may be achieved. Dosages in the range of about 0.01 to about 10 mg/kg of body weight are contemplated, but it would be evident to one of ordinary skill in the art that for particular applications, it may be necessary to use either greater or lesser dosages. Furthermore, because it is possible to generate an immune response to the P. gingivalis functionality, it will be evident to those skilled in the art that the P. gingivalis proteinase, HagA protein, or peptides or functional domains thereof may not be amenable to chronic administration. However, through processes known in the art for mimicking biological activity of proteins through development of peptidomimetics, DNA agents, or other small molecules, repeat administration of active site mimics would be acceptable, including for chronic administration, as needed.

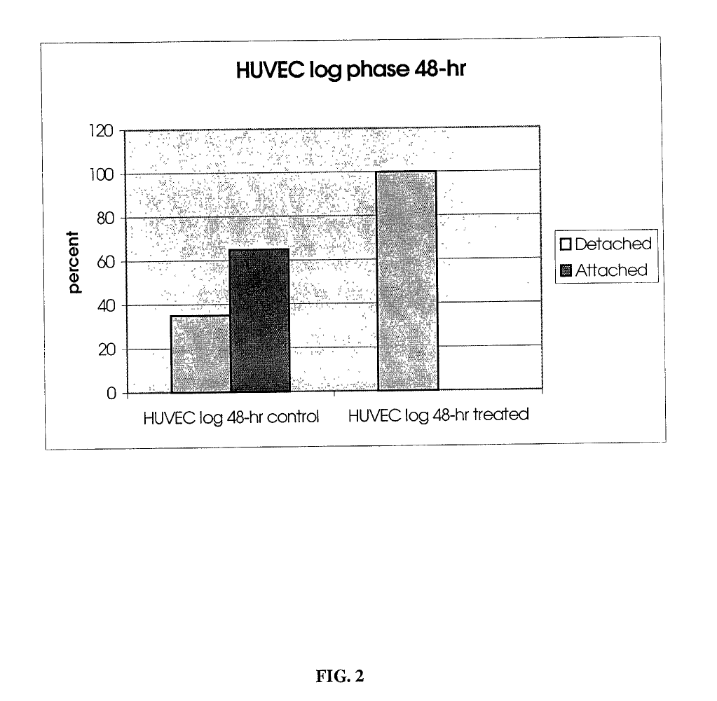

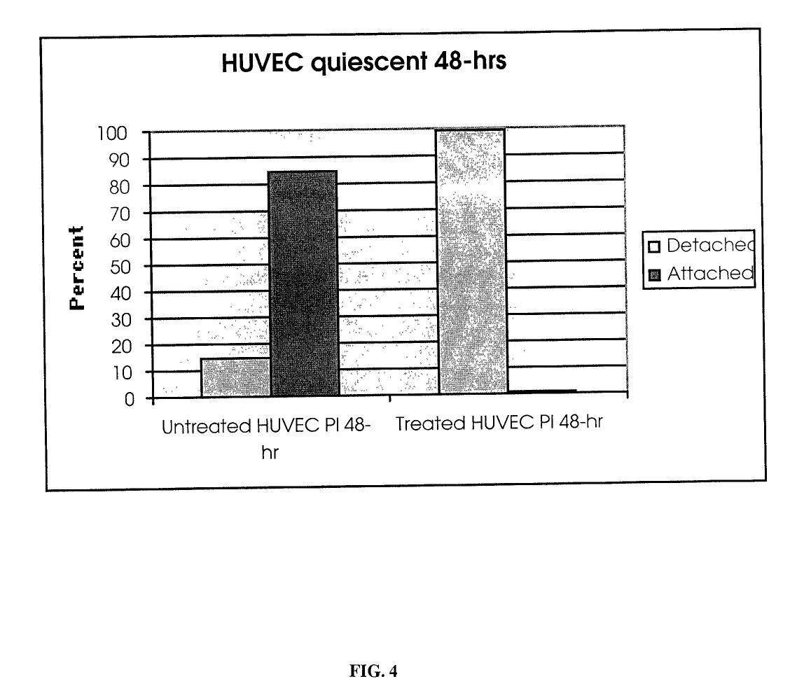

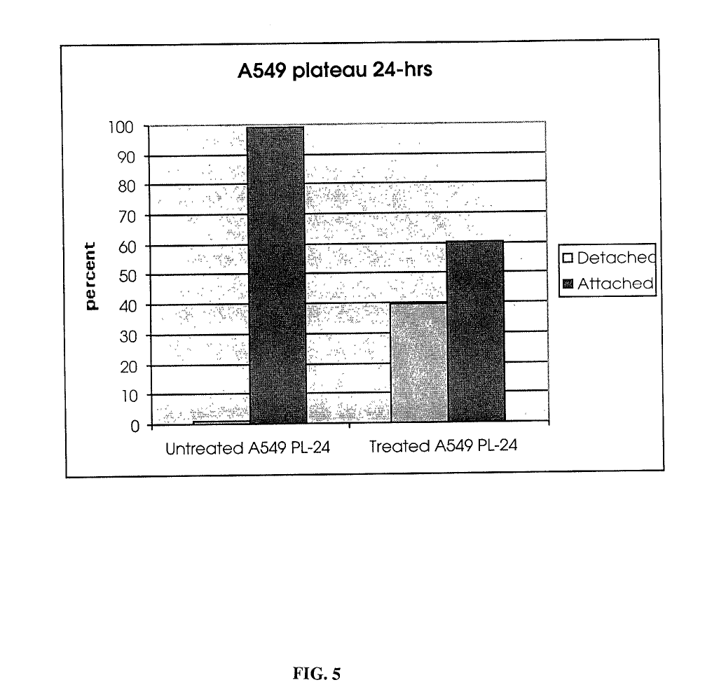

As disclosed in U.S. Pat. No. 5,824,791, FIG. 4, hereby incorporated by reference, the HadA gene 7887 bp molecule, encodes a gene product of 2628 amino acids. Within the gene product, there are four repeat segments, HArep1, HArep2, HArep3, and HArep4. It is predicted that each of these segments provide an active site which may be utilized according to this invention to prevent angiogenesis. The 15 kd protein reported in the literature as being instrumental in the production of necrosis which leads to periodontal disease is located within the HagA sequence between amino acids 683 to 819 in the first repeat and similarly located in the other repeats (see also Han et al., Infect. Immun. Page 4002, FIG. 2). Those skilled in the art will appreciate that the entire HagA molecule may be utilized according to this invention to achieve angiostatic effects. Alternatively, any of the HArep sequences may be utilized, and the 15 kd segment may be utilized according to this invention. Furthermore, based on the present disclosure, those skilled in the art will appreciate that the active site may further be elucidated and employed as a minimal sequence to achieve angiostatic effects. Peptidomimetic compounds may likewise be developed which have equal or greater potency than the active site, and such compounds come within the scope of this invention.

Having generally described this invention, including its best mode, methods of making and use thereof, it will be appreciated by those skilled in the art that in one embodiment of this invention, novel methodology has been disclosed herein for treatment of an angioproliferative disorder which comprises administering to a patient afflicted with such an angioproliferative disorder a pharmaceutically effective amount of P. gingivalis proteinase to exert an angiostatic effect. Antioproliferative disorders in which the present methodology may be applied include, but are not limited to, carcinomas, sarcomas, melanomas, ocular retinopathy, retrolental fibroplasia, psoriasis, angiofibromas, endometriosis, hemangiomas, rheumatoid arthritis, capillary proliferation within atherosclerotic plaques, or a combination of such disorders. In one preferred embodiment, the proteinase is derived from a bacterium, such as Porphyromonas gingivalis, and contains. Furthermore, we have found the method to be effective when the proteinase is related to the HagA gene or other Cys proteinases of P. gingivalis.

It will further be appreciated from the present disclosure that a composition for treatment of an angioproliferative disorder may be prepared by providing a pharmaceutically effective amount of a protease and an excipient for administration to a patient afflicted with an angioproliferative disorder. Excipients known in the art for various modes of administration are known and are not discussed herein in great detail. However, for example, intravenous administration of the composition would naturally lead the skilled artisan to utilize a liquid excipient, such as a saline solution. Oral administration will lead to use of various forms of coating materials to form capsules, tablets or the like, with emphasis on delivery of the active proteins, DNA, peptides or peptidomimetics to the appropriate portion of the digestive tract prior to release thereof, such that destruction of the active components of the composition is minimized or avoided altogether. Furthermore, those skilled in the art have a wide variety of transdermal excipients known in the art to choose from.

As will be appreciated from the present disclosure, a further aspect of this invention includes methods for selectively treating an angioproliferative disorder by contacting the vasculature supplying a biological structure affected by an angioproliferative disorder with an angiostatically effective amount of a proteinase. Due to the known leakyness of vasculature supplying tumors, according to this invention, local or systemic administration of the protease of this invention facilitates contact with the basolateral surface of said vasculature, including the endothelium.

A further aspect of this invention that will be appreciated from the full disclosure hereof includes the ability of the present compositions and methods to potentate the effects of a chemotherapeutically effective agent which comprises co-administering said chemotherapeutically effective agent in the presence of a protease effective to disrupt cell-cell adhesion, cell-matrix adhesion, or both. Such co-administration may be in the form of a covalent complex, an ionic complex, a mixture, simultaneous but separate administration, or administration within a relatively close temporal sequence. Appropriate chemotherapeutic agents include, but are not limited to, doxorubicin, antibodies, and the like, according to methods known in the art.

Those skilled in the art will appreciate that the present disclosure provides general teachings relating to the methods of making and using this invention, including its best mode. The disclosure and example which follow should not be construed as limiting the invention. Rather, the scope of legal protection for this invention should be established through reference to the several claims appended hereto, and equivalents thereof.

### EXAMPLE 1

### Methods for Testing Human Endothelial or Carcinoma Cell Detachment Exposed to P. gingivalis Extract

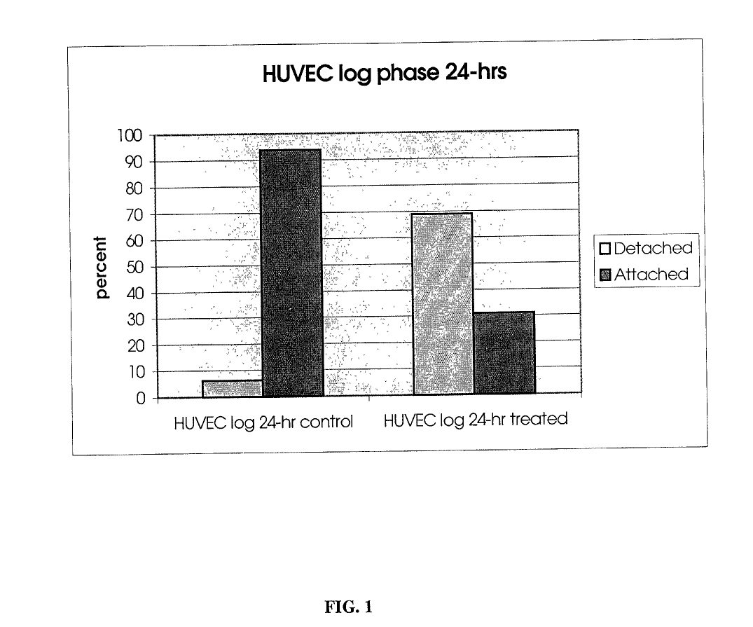

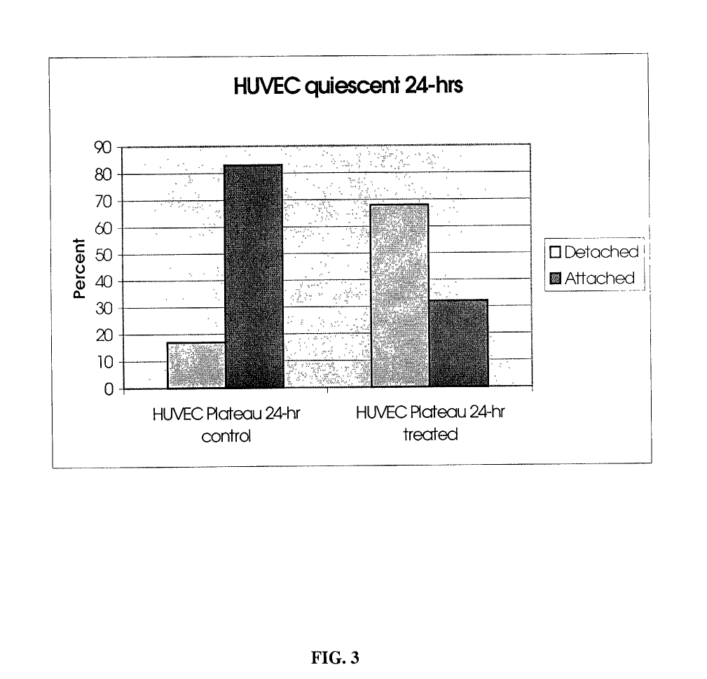

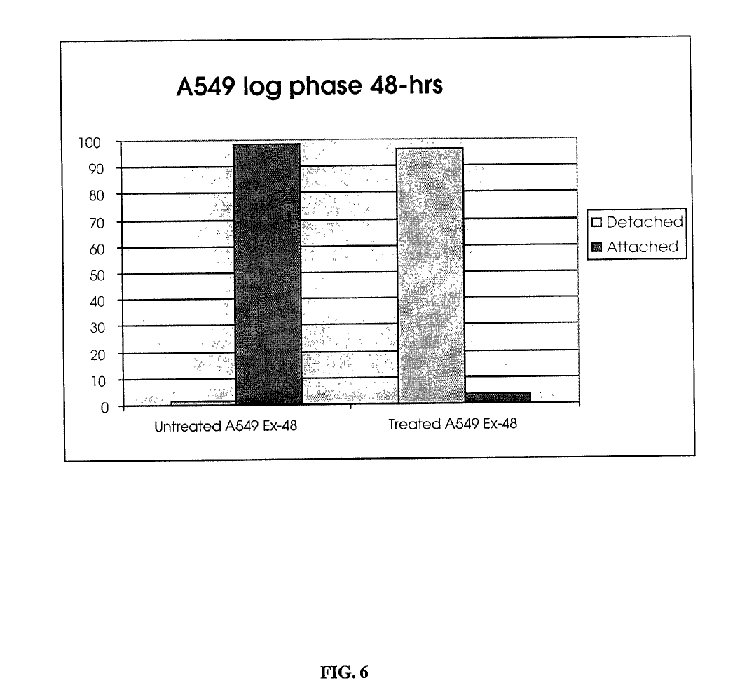

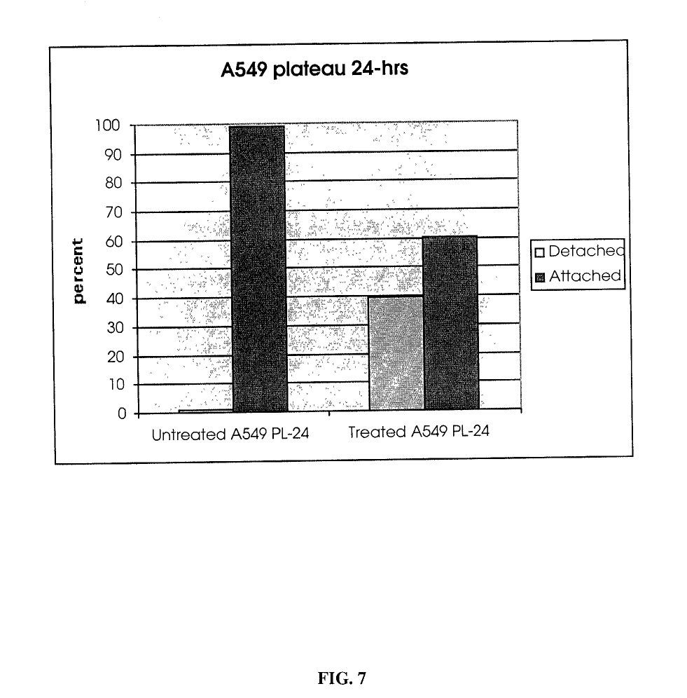

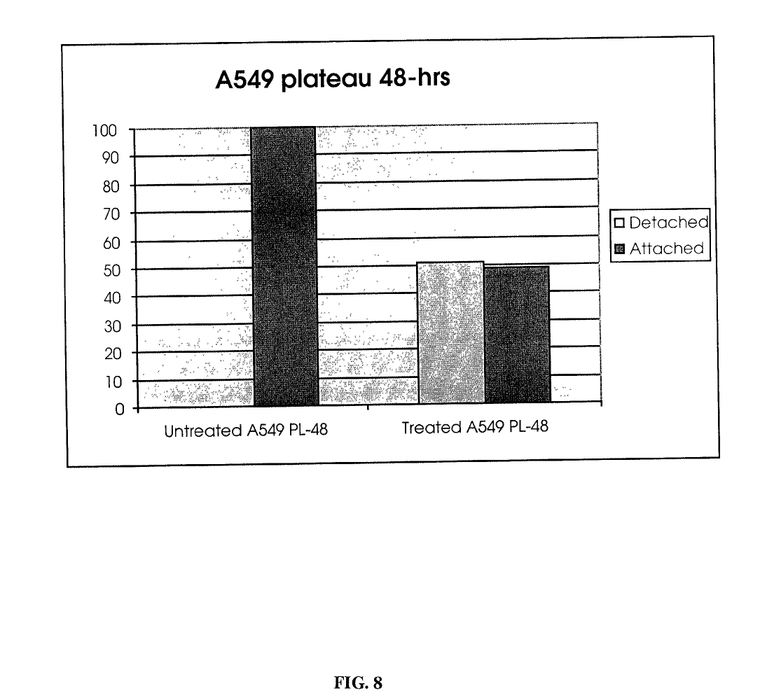

To determine the efficacy of using P. gingivalis protease extracts in the treatment of cancer, two cell lines were tested. Proliferation inhibition was assessed by determining the detachment of tissue culture cells from their substrate. A549 human non-small cell lung carcinoma cell lines and human umbilical vein endothelial cell (HUVEC) lines were used as targets in a cell detachment assay. Exponentially growing (Ex) and quiescent (Plateau) phases of cell culture growth were tested. To obtain extract for activity tests, exponentially grown broth cultures of P. gingivalis strain W83 were pelleted, bacterial cells were resuspended in 50 mM HEPES buffer (pH 7.5) and sonicated on ice for 2 min. Following centrifugation at 14,000 rpm at 4° C., the supernatant was filtered through 0.22 tm non-protein-binding filter (Gelman) and stored at −80° C. until necessary. Protein concentration was measured using Sigma bicinchoninic acid reagent (#B-9643), BSA standard solution and spectrophotometer Shimadzu UV-1201. Six plates per sample with 3 plates added for non-treated control were arranged. 10⁴ cells per 60-mm tissue culture plate were used to seed the sample. Detached cells were counted by collecting the medium after treatment (24 or 48 hrs), washing the attached cells with calcium-free phosphate-buffered saline (PBS) and adding the wash to the medium. The pellet was then resuspended in medium to obtain a countable number on the hemocytometer. The 25-square (0.1 μl volume) count×10⁴ gives the cell count per ml. Two readings were made of each of the three plates. This number represented the detached cell count. The attached cells were trypsinized and collected as above; the plate was washed with PBS and added to the tube. After pelleting and resuspending, two readings were made per each of the three plates to obtain the attached cell count. The mean value of the counts was taken, total number of cells (attached+detached) was obtained and the percentage of detached cells was calculated. Different concentrations of P. gingivalis protein extract were used in the beginning to establish effective concentration to be used throughout. The working concentration was chosen to be 0.4 mg total protein extract per ml culture medium. Detachment from the plastic surface of the petri dish is consistent with the ability of P. gingivalis extract to degrade β1 integrin. FIG. 1 shows a 70% detachment for active Human Umbilical Vein Endothelial Cells (HUVEC) culture cells following treatment with a P. gingivalis extract over a 24-hour period. FIG. 2 shows a 90% detachment of the same cells after 48 hours. FIG. 3 show approximately 68% detachment of quiescent Human (HUVEC) after 24 hours of treatment, whereas the same cells exhibit nearly 100% detachment after 48 hours (see FIG. 4). FIG. 5 shows active A549 human non-small cell lung carcinoma exhibited a 95% detachment rate after 24 hours of treatment with a P. gingivalis extract. After 48 hours, there was no change in the percent detachment of these same cells (see FIG. 6). FIG. 7 shows 40% detachment rate after 24 hours, whereas after 48 hours the detachment rose to greater than 50%. Anti-angiogenic and tumor-reducing activity correlates with the degree of detachment of endothelial and carcinoma cells. The graphs demonstrate strong proliferation inhibition of the endothelial and cancer tissue culture cells in both 24 and 48-hour treatments. Demonstrating detachment of up to 96% of the cells (A549) or 98% (HUVEC) can thus be considered pertinent activity. This is for total protein where the fraction of the active ingredient is small. Hence its activity is conceivably very high. Both exponentially growing (“log”) and stationary phase (“plateau”) quiescent cultures showed differences in ability to remain attached following treatment.

### EXAMPLE 2

### Method of Demonstrating Detachment Associated with PrtP Protease from P. gingivalis

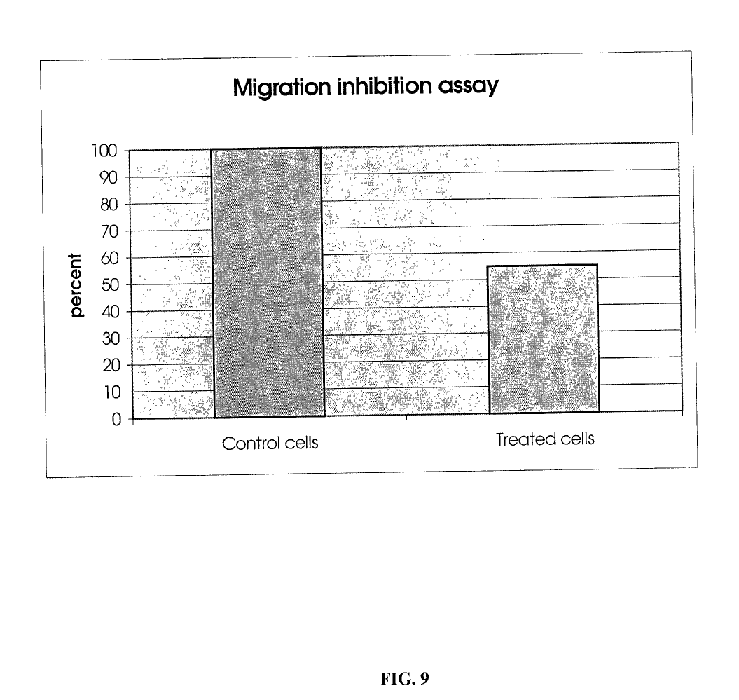

To demonstrate that the protease PrtP isolated from P. gingivalis is responsible for the detachment observed in Example 1, three sample were set up and applied to A549 lung carcinoma cells. Single P. gingivalis protein, the PrtP protease, was expressed in Bacteroides fragilis, a species related to P. gingivalis, but which does not express PrtP, for the purpose of further chromatographic purification. Therefore, treatment of carcinoma cells was performed with extract of B. fragilis containing PrtP and compared to the same treatment with a wild-type B. fragilis host. The difference between the treatments was limited in this way to the presence/absence of P. gingivalis PrtP protease only. The strains were grown in BHIS broth (per liter, 37 g Brain Heart Infusion (Difco), 1 g L-Cysteine (Sigma), 5×10⁻⁴% hemin, 0.2% NaHCO₃ in an anaerobic chamber with an atmosphere of 5% CO₂, 10% H₂, and 85% N₂). Agar (1.5%) was added for solid medium. P. gingivalis W83 was grown on Trypticase soy agar (BBL Microbiology Systems, Cockeysville, Md.) supplemented with sheep blood (5%), hernin (5 mg/ml), and menadione (5 mg/ml). When broth-grown P. gingivalis was required, cultures were grown in Todd-Hewitt broth (BBL Microbiology Systems) supplemented with hemin (5 μg/ml), menadione (5 μg/ml), and glucose (2 mg/ml) anaerobically. Normal Bacteroides fragilis was used as a control. As can be seen in FIG. 9, detachment of cells exposed to both B. fragilis with PrtP and P. gingivalis were nearly equal, whereas those cells exposed to the control exhibited minimal detachment. This study provides direct evidence that PrtP is active in cell proliferation inhibition.

### EXAMPLE 3

### Migration Inhibition Assay

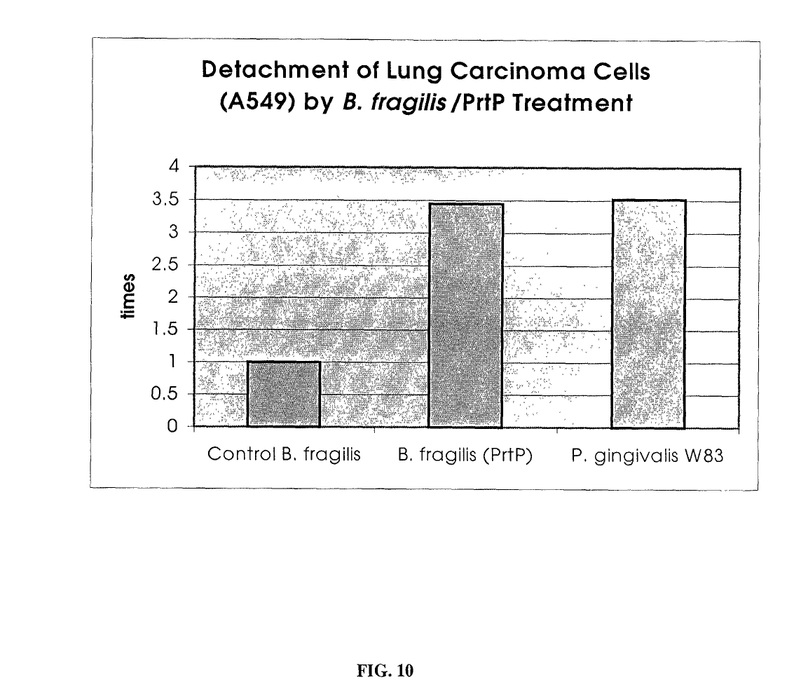

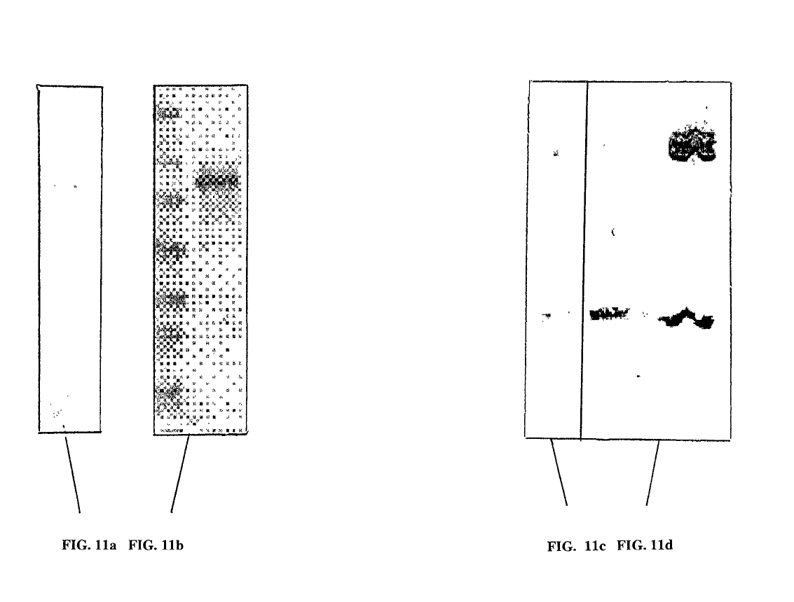

To demonstrate that P. gingivalis extract exerts anti-angiogenic effects, as opposed to general inhibition of cell proliferation, the following assay was performed. P. gingivalis were produced and homogenized to obtain an extract as described in Example 1.. At 0.4-mg total protein/ml, human vascular endothelial cell migration in a standard in vitro assay known in the art to reflect angiostatic and anti-tumor activity, was reduced by 45%, (mean value of 2 experiments) (see FIG. 10). In addition, at 48 hours, detachment of 85% of the cells was observed (not shown). Since total cell protein was used, where the fraction of the active ingredient is small, this experiment demonstrates that the angiostatic activity of the P. gingivalis proteinase is high.

### EXAMPLE 4

### Identification of Epithelial Cell Ligands of Hemagglutinin A

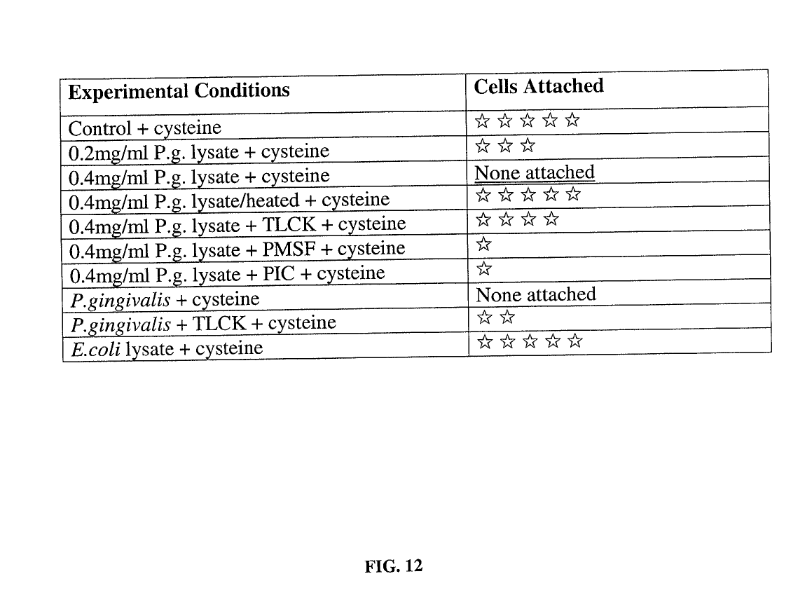

In order to determine if HagA interacts directly with host cell components, a functionally active fragment of HagA was produced in E. coli using the E. coli expression vector, pET19b (Novagen). In this system, purification was achieved by fusing a histidine tag to the HagA fragment and by affinity purification of the fusion protein on a Ni²⁺ column. For this, oligonucleotides were designed flanking 2 HArep sequences to include the active site of hemagglutination as disclosed in U.S. Pat. No. 5,824,791 (herein incorporated by reference) and to include restriction sites for ligation of the fragment into the expression vector, pET19b. The 3 kb PCR product was first cloned into pT7Blue vector (Novagen), digested with NdeI and XhoI, and the coding sequence was directionally subcloned into pET19b, which had been digested with the same enzymes and CIP-treated. Using PCR with a mixed pair of primers T7 (from vector) and ST2/3′ (from insert), transformants in E. coli Novablue (Novagen) were screened for an insert in the proper orientation. One such clone, pEKS5, was chosen for further work and was transformed into E. coli BL21(DE3), an expression strain (Novagen). After induction with 1 mM IPTG, cells were lysed and the lysate was applied to an activated His. Bind resin affinity chromatography column. Elution with 1 M imidazole-containing buffer produced a single protein species with an apparent molecular mass of ˜100 kDa (FIG. 11A). After transfer onto a nitrocellulose membrane, the protein was probed with anti-HagA antibody, 61BG1.3 and its authenticity was confirmed. (See FIG. 11B). The purified recombinant Hag A peptide was then tested for binding to cell components of two human cell lines using the Far Western immunoblot. For this assay, KB oral epithelial cells and human umbilical cord endothelial cells (HUVEC) were grown and lysed in hypotonic buffer containing a cocktail of mammalian proteinase inhibitors. The cell lysates were loaded on SDS-PAGE gels, transferred to nitrocellulose membranes, blocked with Carnation dry fat-free milk in TBS, and overlaid with 0.5 μg/ml of purified recombinant HagA. After 3 hours of incubation at ambient temperature followed by washing, the membranes were treated first with anti-HagA Mab and secondly with anti-mouse AP conjugate. The HagA peptide was found to bind intensely to two proteins, 60 kDa and 65 kDa in size, present in both epithelial (FIG. 5, lane 1) and endothelial (lane 3) cells (see FIGS. 12A and B). The HagA peptide also bound to two heavy protein species, >200 kDa, present in endothelial cells. These results demonstrate that HagA binds to and interacts with one or more proteins present in host cells and suggest the in vivo existence of a protein complex between HagA and endothelial as well as epithelial proteins.

### EXAMPLE 5

### Maintenance of Tissue Culture Cell Lines (HUVEC #ECV-304, HUH7)

Tissue culture cells grown in T-75 flasks at 37° C. in DMEM (Pen/Strep) in a CO₂ incubator were subjected to 8 ml of trypsin-EDTA and incubated at 37° C. for 10-15 minutes for detachment. Trypsinized cells were transferred (with 2×10 ml DMEM) to 50 ml culture tube and centrifuged at 1K rpm for 10 seconds. Supernatant was removed and cells were washed with 8 ml Ca-free PBS. Washed cells were centrifuged at 1K rpm for 10 seconds and excess wash was removed. Cells were resuspended in 20 ml of DMEM (Pen/Strep) media and transferred to new T-75 flasks for incubation at 37° C. in 5% CO₂.

### EXAMPLE 6

### Immunofluorescent Microscopy of HUH7 Human Hepatoma Cells Treated with P. gingivalis Extract

HUH7 cells were incubated with P. gingivalis extract (0.8 mg protein per ml of medium) for 20 h at which time the cells were washed three times with phosphate buffered saline (PBS) and then fixed in 4% paraformaldehyde in PBS for 30 min at room temperature. This was followed by washing twice in PBS and quenching in NH₄Cl (50 mM)/0.3% Tween 20/PBS for 10 min at room temperature. After quenching, the HUH7 were washed two times in PBS. The primary antibodies were rabbit anti-human occludin (Zymed Laboratories #71-1500) and rabbit anti-human pan cadherin (Sigma Chemical Co., St. Louis, Mo. #C3678). They were diluted 1/50 in PBS/5% normal goat serum/0.3% Tween 20 and applied to the cells for 2 h at room temperature. The HUH7were then washed four times in PBS for 5 min each time. The secondary antibodiy [rhodamine-conjugated goat anti-rabbit (Sigma)] was applied for 1 h at room temperature. The HUH7 were then washed twice with PBS before mounting with Fluoromount-G (Southern Biotechnology Associates, Inc., Birmingham, Ala.) onto glass microscope slides and sealing with nail polish. Images were viewed using an Olympus IX70 deconvolution microscope and Deltavision software (Applied Precision, Inc., Wepahah, Wash.).

### EXAMPLE 7

### Proliferation Inhibition of HUH7 by P. gingivalis and E. coli Extracts, and by Live P. gingivalis Cells in the Presence/absence of Inhibitors

Freshly collected whole P. gingivalis cells were used for 20-hour treatment at a density of 2×10¹⁰ bacteria per ml DMEM (antibiotic-free). FIG. 12 represents the proliferation inhibition of HUH7 by P. gingivalis and E. coli extracts, and by live P. gingivalis cells in the presence/absence of inhibitors (five stars: all cells remain attached, no proliferation inhibition). L-Cysteine was always present at concentration of 5 mM to stabilize the antiangiogenic activity. The proliferation inhibition property of P. gingivalis extract and whole cells is clearly demonstrated on human hepatoma cell line (HUH7).

### EXAMPLE 8

### Reactivity with Anti-Occludin Antibody

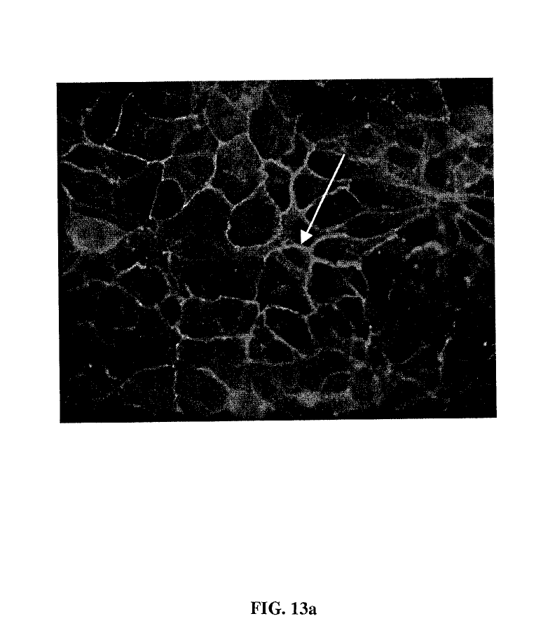

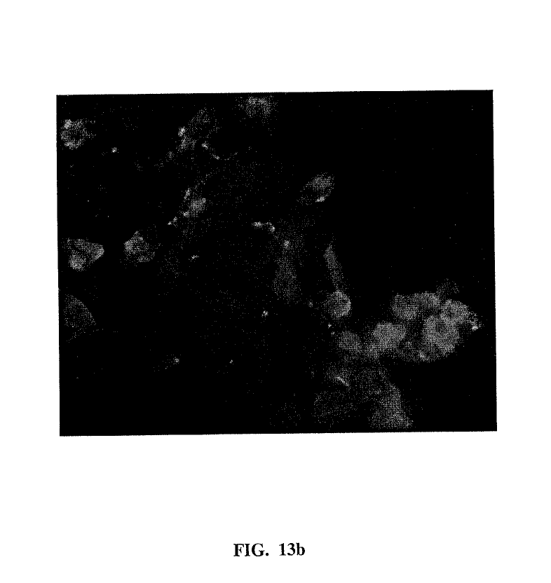

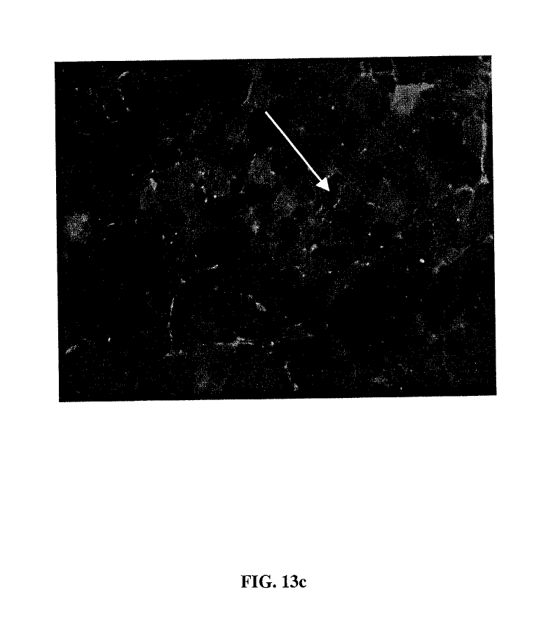

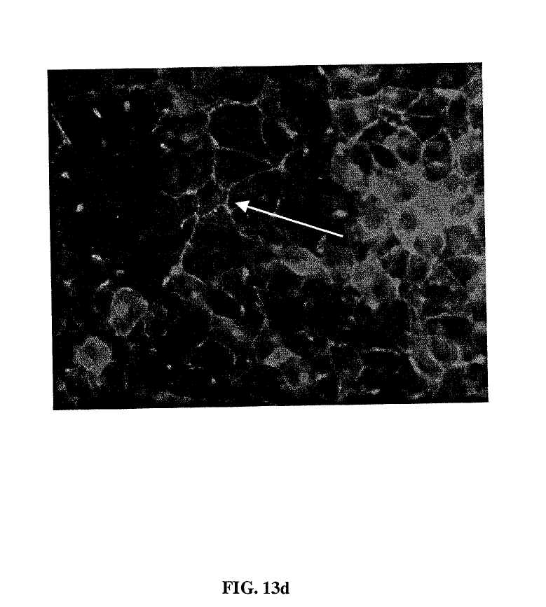

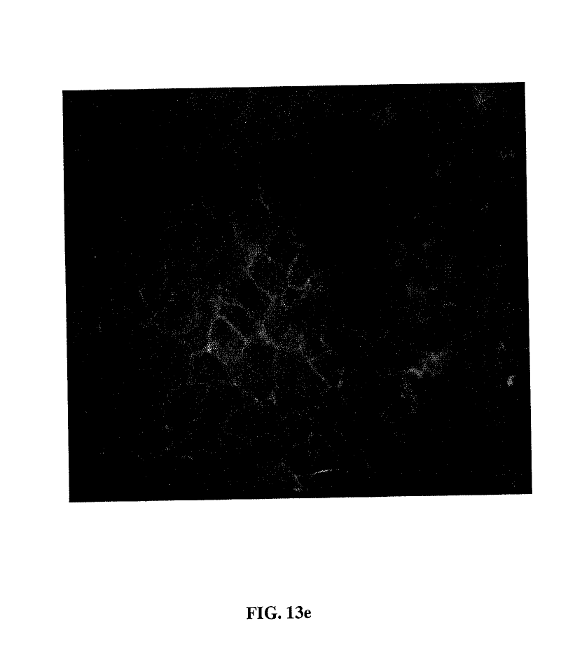

Occludin-stained junctions degraded upon treatment with P. gingivalis extract. TLCK presence or heat-inactivation of P. gingivalis extract abolishes the activity. E. coli extract control treatment exhibited no activity. FIG. 13a shows non-treated HUH7 FIG. 13b shows HUH7 cells after treatment with P. gingivalis extract; FIG. 13c shows HUH7 cells after treatment with P. gingivalis extract in the presence of inhibitor TLCK; FIG. 13d shows HUH7 following treatment with heat-treated P. gingivalis extract; and FIG. 13d shows HUH7 cells treated with E. coli extract and demonstrates that E. coli does not effect the occludin network. The data from immunoflourescent staining of junctional molecules from HUH7 cells confirms the capacity of P. gingivalis extract to disrupt the intracellular network by degrading the border consisting of cell adhesion molecules (CAMs).

### EXAMPLE 9

### Reactivity with Anti-Pan Cadherin Antibody

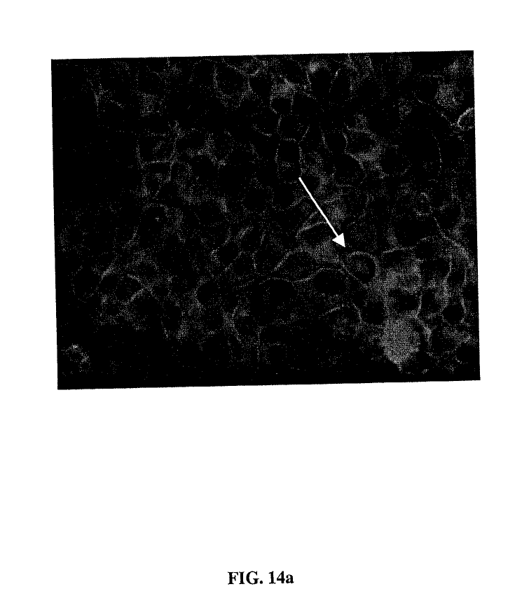

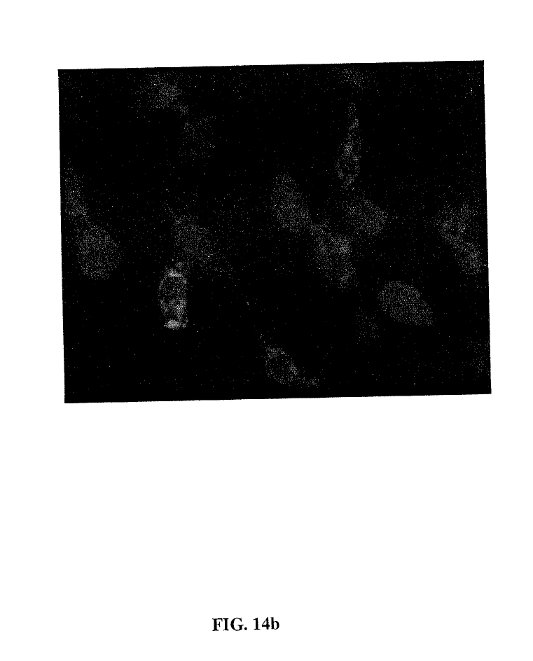

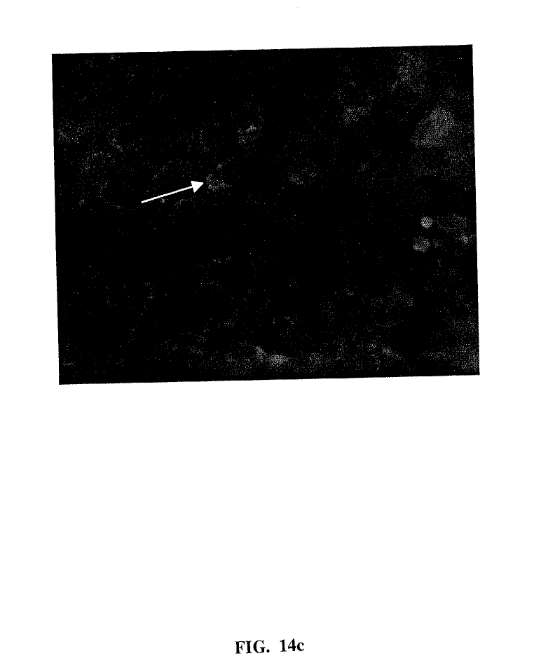

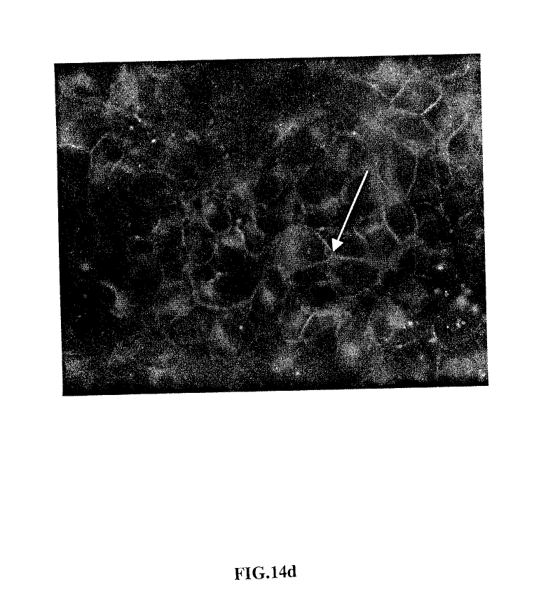

Cadherin-stained junctions were degraded upon treatment with P. gingivalis extract. TLCK presence or heat-inactivation of P. gingivalis extract abolished the activity. FIG. 14a shows control, non-treated HUH7 hepatoma cells; FIG. 14b shows HUH7 cells following treatment with with P. gingivalis extract; FIG. 14c shows HUH7 cells after treatment with P. gingivalis extract in the presence of inhibitor TLCK; and FIG. 14d shows HUH7 cells after treatment with heat-inactivated P. gingivalis extract. The proliferation. As stated above, the data from immunoflourescent staining of junctional molecules from HUH7 cells confirms the capacity of P. gingivalis extract to disrupt the intracellular network by degrading the border consisting of cell adhesion molecules. Thus both examples 8 and 9 demonstrate that the unique activity of the present disclosed extract can be utilized for tumor disintegration.

### EXAMPLE 10

### Fractioning of Bacterial Culture Liquor Proteins

In order to partially purify the CAM-degrading activity, fractional precipitation of secreted P. gingivalis proteins from spent culture liquor was achieved using ammonium sulfate. Broth culture grown in an anaerobic chamber was centrifuged for 20 minutes at a speed of 8000 rpm. Next it was filtered with a 0.2 μm filter (Nalgene) to remove any remaining cells. To saturate to 60%, 36.1 g of (NH₄)2SO₄ were dissolved in every 100 ml of culture liquor. The solution was left stirring overnight at 4° C. and collected the next day by centrifuging for 20 min. at 8000 rpm. Precipitated proteins were collected from 6 liters of P. gingivalis W83 spent culture medium. The protein pellet was resuspended in a 20 ml solution of 50 mM Tris.HCl (pH 7.5). The solution was dialyzed (Pierce SnakeSkin tubing, 7 kDa MWCO) against 50 mM Tris.HCl overnight at 4° C. The dialysis was repeated with fresh buffer. Dialyzed solution was filtered with an Acrodisc® syringe filter (0.2 μm) and then concentrated using a Centriprep 10 (Amicon) for a total of an hour and a half. After concentration, the solution was aliquoted, the protein concentration was determined using the BCA assay (Sigma B-9643), then stored at −80° C.

### EXAMPLE 11

### Proliferation Inhibition of HUVEC Polarized Cell Line. HUVEC #ECV-304 Treatment with P. gingivalis Fractions

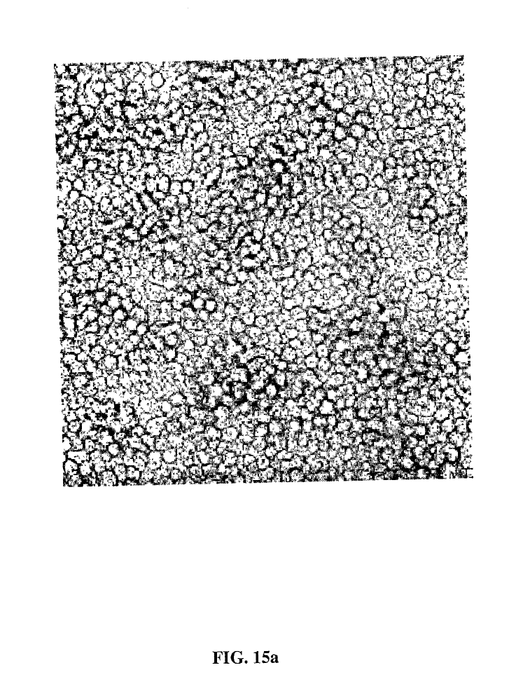

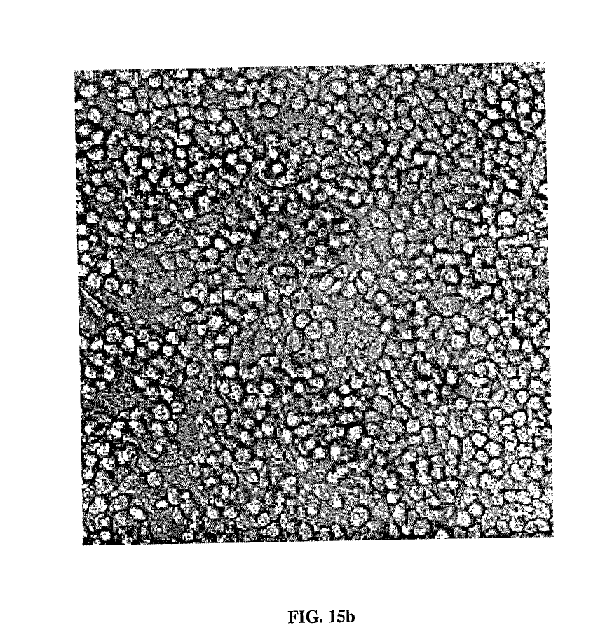

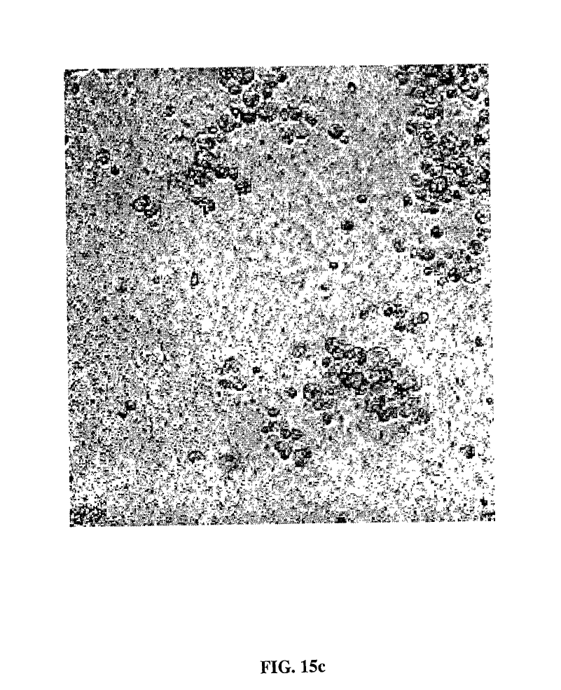

Polarized endothelial cells cultured on porous membrane inserts (Transwell, Corning Costar Corp., Cambridge, Mass.) were used as an in vitro model for studying antiangiogenic activities and to test for differential activity from both sides of the endothelium. Six hundred μl (for 24-well plate) or 2.6 ml (for 6-well plate) of DMEM medium (Penn/Strep) were added to the lower chamber of tissue culture plates. Vascular endothelial cells were seeded into Coming Costar Transwell inserts in volumes of 0.1 ml medium (24-well plate) or 1.5 ml medium (6-well plate) in the upper chamber. Cultures were grown to confluence in a CO₂ incubator at 37.0° C. before being treated. The proteins were added to the upper or lower chambers at a final concentration of 0.8 mg/ml. Then the cultures were incubated for 4 days in CO₂ incubator. Similar results were obtained using whole P. gingivalis cells (data not shown). FIG. 15a shows a control, untreated polarized human endothelial cells ECV-304; FIG. 15b shows polarized ECV-304 cells treated lumenally with 60% fraction of P. gingivalis culture liquid proteins; and FIG. 15c shows polarized ECV-304 cells following treatment basolaterally with 60% fraction of P. gingivalis culture liquid proteins. In each experiment the polarized endothelial cell layer was treated from either the apical or. basolateral side with identical concentration of protein preparations. As seen in the optical micrographs in FIGS. 15a, b and c. at a point where complete destruction was observed from basolateral application of ammonium sulfate—precipitated proteins or whole bacteria, no damage was observed in the cultures with lumenal (apical) application of same preparations. These data strongly support the conclusions tha antiangiogenic activity is partially purified from P. gingivalis secreted proteins as 60-% fraction of ammonium sulfate-precipitated culture liquor proteins; and the targeting of this activity toward the basolateral, extravascular side of the vasculature is specifically beneficial for degradation of the endothelial vascular cell layer in abnormally leaky tumor vessels. In addition to antiangiogenic activity, immunofluorescent and proliferation inhibition studies with human cancer cell lines (hepatoma and lung carcinoma) strongly suggest the utility of this P. gingivalis-associated activity for disintegration of extravascular tumor tissues, i.e. direct tumor-disintegration activity exists. Using the same abnormal openings to access both the basolateral side of the tumor vasculature and the surrounding tumor tissue brings double benefit to the proposed treatment.

In light of foregoing evidence, it is apparent that the P. gingivalis proteinase may be utilized as a vascular endothelial cell migration inhibitor and as an angiostatic pharmaceutical agent. Furthermore, while at present there does not appear to be any known therapeutic protocol based on selective degradation of cell-cell and cell-matrix adhesion molecules in tumors and a large number of other diseases, the present invention provides a new method of disease treatment for such pathologies. It is further predictable, based on the disclosure provided herein, that other proteins, known or yet to be discovered which exhibit similar angiostatic activity, may be used according to the methods of this invention. Furthermore, combinations of such molecules may also be used according to the methods of this invention.

| Experimental Conditions | Cells Attached |
|---|---|
| Control + cysteine | ☆☆☆☆☆ |
| 0.2 mg/ml P.g. lysate + cysteine | ☆☆☆ |
| 0.4 mg/ml P.g. lysate + cysteine | None attached |
| 0.4 mg/ml P.g. Lysate/heated + cysteine | ☆☆☆☆☆ |
| 0.4 mg/ml P.g. lysate + TLCK + cysteine | ☆☆☆☆ |
| 0.4 mg/ml P.g. lysate + PMSF + cysteine | ☆ |
| 0.4 mg/ml P.g. lysate + PIC + cysteine | ☆ |
| P. gingivalis + cysteine | None attached |
| P. gingivalis + TLCK + cysteine | ☆☆ |
| E. coli lysate + cysteine | ☆☆☆☆☆ |

What is claimed is:

1. A method for treatment or prevention of an angioproliferative condition which comprises administering to a patient experiencing said angioproliferative condition a pharmaceutically effective amount of a proteinase to exert an angiostatic effect.

2. The method according to claim 1 wherein said angioproliferative condition is a carcinoma, sarcoma, melanoma, ocular retinopathy, retrolental fibroplasia, psoriasis, angiofibromas, endometriosis, hemangioma, rheumatoid arthritis, capillary proliferation within atherosclerotic plaque, or a combination of such disorders.

3. The method according to claim 1 wherein said proteinase is derived from a bacterium.

4. The method according to claim 3 wherein said bacterium is Porphyromonas gingivalis.

5. The method according to claim 4 wherein said protease is PrtP, HagA, other cysteing proteinase, a HagArep peptide, a fragment or active site thereof, or DNA.

6. A composition for treatment or prevention of an angioproliferative condition comprising a pharmaceutically effective amount of a proteinase and an excipient for administration to a patient afflicted with said angioproliferative disorder.

7. The composition according to claim 6 wherein said angioproliferative condition is a carcinoma, sarcoma, melanoma, ocular retinopathy, retrolental fibroplasia, psoriasis, angiofibromas, endometriosis, hemangioma, rheumatoid arthritis, capillary proliferation within atherosclerotic plaque, or a combination of such disorders.

8. The composition according to claim 6 wherein said proteinase is derived from a bacterium.

9. The composition according to claim 8 wherein said bacterium is Porphyromonas gingivalis.

10. The composition according to claim 9 wherein said protease is PrtP, HagA, other P. gingivalis proteinase, a HagArep peptide, a fragment or active site thereof, or DNA

11. A method for selectively treating an angioproliferative condition which comprises contacting the vasculature supplying a biological structure affected by said angioproliferative condition with an angiostatically effective amount of a protease.

12. The method according to claim 11 wherein said proteinase is contacted with the basolateral surface of said vasculature.

13. The method according to claim 11 wherein said angioproliferative condition is a carcinoma, sarcoma, melanoma, ocular retinopathy, retrolental fibroplasia, psoriasis, angiofibromas, endometriosis, hemangioma, rheumatoid arthritis, capillary proliferation within atherosclerotic plaque, or a combination of such disorders.

14. The method according to claim 12 wherein said protease is derived from a bacterium.

15. The method according to claim 12 wherein said bacterium is Porphyromonas gingivalis.

16. The method according to claim 15 wherein said protease is PrtP, HagA, other proteinase a HagArep peptide, a fragment or active site thereof, or DNA.

17. A method for potentiating the effects of a chemotherapeutically effective agent which comprises co-administering said chemotherapeutically effective agent in the presence of a protease effective to disrupt cell-cell adhesion, cell-matrix adhesion, or both.

18. A method for preventing the implantation or sustenance of a fertilized ovum which comprises administering an angiostatically effective amount of a proteinase to a person in whom such preventing is required, sufficient to prevent formation of new vasculature required for implantation or sustenance of said fertilized ovum.

19. A method for inhibiting vascular endothelial cell migration which comprises contacting vascular endothelial cells with a molecule selected from the group consisting of cysteine proteinase, HagA protein, HagA peptide, HagA-specific enzymatic activity, HagA active site mimetic, HagA analog, and combinations thereof or DNA
A method for reducing cell-cell adhesion, cell-matrix adhesion, or both, which comprises contacting cells, matrix or both with an effective amount of a molecule selected from the group consisting of a cysteine proteinase, HagA protein, HagA peptide, HagA-specific enzymatic activity, HagA active site mimetic, HagA analog, and combinations thereof or DNA
